(19) United States
(12) Patent Application Publication
(10) Pub. No.: US 2010/0192947 A1
(43) Pub. Date: Aug. 5, 2010

(54) ANESTHETIC DELIVERY SYSTEM AND METHODS OF USE

(76) Inventors: Jeff MANDEL, Media, PA (US); Marcos AMARO, Philadelphia, PA (US)

Correspondence Address: Pearl Cohen Zedek Latzer, LLP, 1500 Broadway, 12th Floor, New York, NY, 10036, US

(21) Appl. No.: 12/365,205

(22) Filed: Feb. 4, 2009

(51) Int. Cl. A61M 16/22; A61M 16/01

(52) U.S. Cl. 128/203.14; 128/203.28; 128/205.28

(57) ABSTRACT

This invention relates to systems and methods of delivering anesthetic to a subject. Specifically, the invention relates to systems and methods of delivering an anesthetic to a subject while being able to regulate and recover the ensethetic gas from the air upon expiration.

### FIELD OF INVENTION

This invention is directed to systems and methods of delivering anesthetic to a subject. Specifically, the invention is directed to systems and methods of delivering an anesthetic to a subject while being able to regulate and recover the ensethetic gas from the air upon expiration.

### BACKGROUND OF THE INVENTION

Atrial fibrillation accounts for over 400,000 admissions annually in the US, and no less than $6 billion in direct costs. The incidence is expected to increase 2-4 fold by 2050. Increased utilization of cardioversion (CVN) has been demonstrated to reduce direct cost by $1400/episode. Most patients require sedation for CVN, typically with IV sedatives.

Sevoflurane and other volatile agents have advantages over IV agents, including less hypotension and respiratory depression. Sevoflurane and other volatile agents are administered by out-of-circle vaporizers; such systems are cumbersome and inefficient for brief anesthetics in ad hoc locations.

Several factors enter into determining the cost of an anesthetic. These factors include the cost of acquisition and preparation, cost of wasted drug, cost of adverse effects, and cost of treatment failures. Sevoflurane is suitable for inhaled inductions and is used extensively for this purpose in pediatric patients. A limitation of sevoflurane and other similar anesthetics (e.g. Desflurane), is that the drug is relatively expensive. Although this expense can be somewhat reduced during maintenance anesthesia by reducing the fresh gas flow to about 1 L/min, which is the lowest currently recommended rate in the United States and several other countries, however, during induction—flows near about 10 L/min are required to rapidly fill the rebreathing system and to compensate for absorption of the anesthetic into the patient's lungs and circulation. The result is that an inhaled induction with sevoflurane can contribute substantially to the total cost of anesthetic drugs, especially during short procedures.

A marked decrease in both personal and environmental pollution with anaesthetic gases as well as in anesthetic gas costs is necessary with anaesthesia machines, especially when relatively expensive albeit effective anesthetic gasses are used for induction.

### SUMMARY OF THE INVENTION

In one embodiment, the invention provides a method for administering an anesthetic to a patient, the method comprising: streaming the anesthetic into an apparatus (10) comprising: a canister (20) having an input lumen port (21) and an output lumen port (22), wherein the canister (20) comprises a CO₂ scavenging means, the input lumen port (21) operably linked to a breathing bag (30) and the output lumen port (22) having three openings therein; an axial opening (223), a first radially disposed opening (221), and a second radially disposed opening (222), wherein the first and second radially disposed openings are diametrically opposed; a flow diverting/relief valve (50) operably linked to the first radial opening (221) in the output lumen port (22); an anesthetic gas supply (60), operably linked to the second radially disposed opening (222) in the output lumen port (22); a subject interface means (70), operably linked to the axial opening (223); and a volatile gas scavenging means (500) having a proximal end (501) and a distal end (502), the proximal end (501) being operably coupled to the diverter valve (50); and the distal end (501) operably linked to the output port (22) of the apparatus; venting the anesthetic gas to the output port (22) of the apparatus (10) for inspiration by the subject; venting expirated air to the CO₂ scavenging means (20) of the apparatus, thereby cleaning the expirated air of CO₂ to be re-circulated to the subject; and diverting expirated air to a volatile gas scavenging means (500), thereby absorbing at least some of the volatile anesthetic gas in the exhaled air.

In another embodiment, the invention provides an apparatus comprising: a canister (20) having an input lumen port (21) and an output lumen port (22), wherein the canister (20) comprises a CO₂ scavenging means, the input lumen port (21) operably linked to a breathing bag (30) and the output lumen port (22) having three openings therein; an axial opening (223), a first radially disposed opening (221), and a second radially disposed opening (222), wherein the first and second radially disposed openings are diametrically opposed; a flow diverting/relief valve (50) operably linked to the first radial opening (221) in the output lumen port (22); an anesthetic gas supply (60), operably linked to the second radially disposed opening (222) in the output lumen port (22); a subject interface means (70), operably linked to the axial opening (223); and a volatile gas scavenging means (500) having a proximal end (501) and a distal end (502), the proximal end (501) being operably coupled to the diverter valve (50); and the distal end (501) operably linked to the output port (22) of the apparatus.

Other features and advantages of the present invention will become apparent from the following detailed description and figures. It should be understood, however, that the detailed description and the figures, while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings in which like reference designators are used to designate like elements, and in which:

FIG. 1 shows an embodiment of the apparatus comprising: a canister (20) having an input lumen port (21) and an output lumen port (22), wherein the canister (20) comprises a CO₂ scavenging means, the input lumen port (21) operably linked to a breathing bag (30) and the output lumen port (22) having three openings therein; an axial opening (223), a first radially disposed opening (221), and a second radially disposed opening (222), wherein the first and second radially disposed openings are diametrically opposed; a flow diverting/relief valve (50) operably linked to the first radial opening (221) in the output lumen port (22); an anesthetic gas supply (60), operably linked to the second radially disposed opening (222) in the output lumen port (22); a subject interface means (70), operably linked to the axial opening (223);

FIG. 2 shows an embodiment of a liquid anesthetic vaporizer;

FIG. 3 shows an embodiment of the volatile gas scavenging means, and

FIG. 4 shows another embodiment of the apparatus comprising: a canister (20) having an input lumen port (21) and an output lumen port (22), wherein the canister (20) comprises a CO₂ scavenging means, the input lumen port (21) operably linked to a breathing bag (30) and the output lumen port (22) having three openings therein; an axial opening (223), a first radially disposed opening (221), and a second radially disposed opening (222), wherein the first and second radially disposed openings are diametrically opposed; a flow diverting/relief valve (50) operably linked to the axial opening (223) in the output lumen port (22); an anesthetic gas supply (60), operably linked to the first radially disposed opening (222) in the output lumen port (22); a subject interface means (70), operably linked to the first radial opening (221), with a volatile gas scavenging means (500) disposed therebetween

### DETAILED DESCRIPTION OF THE INVENTION

This invention relates in one embodiment to systems and methods of delivering anesthetic to a subject. In another embodiment, the invention is directed to systems and methods of delivering an anesthetic to a subject while being able to regulate and recover the ensethetic gas from the air upon expiration.

In one embodiment, provided herein is a method for administering an anesthetic to a patient, the method comprising: streaming the anesthetic into an apparatus (10) comprising: a canister (20) having an input lumen port (21) and an output lumen port (22), wherein the canister (20) comprises a CO₂ scavenging means, the input lumen port (21) operably linked to a breathing bag (30) and the output lumen port (22) having three openings therein; an axial opening (223), a first radially disposed opening (221), and a second radially disposed opening (222), wherein the first and second radially disposed openings are diametrically opposed; a flow diverting/relief valve (50) operably linked to the first radial opening (221) in the output lumen port (22); an anesthetic gas supply (60), operably linked to the second radially disposed opening (222) in the output lumen port (22); a subject interface means (70), operably linked to the axial opening (223); and a volatile gas scavenging means (500) having a proximal end (501) and a distal end (502), the proximal end (501) being operably coupled to the diverter valve (50); and the distal end (501) operably linked to the output port (22) of the apparatus; venting the anesthetic gas to the output port (22) of the apparatus (10) for inspiration by the subject; venting expirated air to the CO₂ scavenging means (20) of the apparatus, thereby cleaning the expirated air of CO₂ to be re-circulated to the subject; and diverting expirated air to a volatile gas scavenging means (500), thereby absorbing at least some of the volatile anesthetic gas in the exhaled air.

In another embodiment, the anesthetic gas used in the methods and devices described herein for cardioversion (CVN) or atrial fibrilation, or other similar brief painful procedure comprises Sevofluorane or Desflurane.

In one embodiment, the device (10) is used in cardioversion, atrial fibrilation or other, brief painful procedures where the reduction of pain by the subject is desired.

In another embodiment, the Carbon Dioxide (CO₂) scavenging means used in the canister described herein (20) and used in the methods described, comprises soda lime. In one embodiment, the anesthetic gas scavenging means (500) used in the methods described herein and the device provided is shown in FIG. 3.

As shown in FIG. 3, the volatile scavanging means (500) are comprised of a cylinder (500) having an input port (501) and at least on output port (502). In one embodiment, two foam discs (506) allow for the passage of a carrier mixture into a medium containing a high surface area compound which has high affinity to the volatile gas sought to be absorbed. This compound may be a combination of compounds such as activated charcoal (504), high-cilica zeolites (505), either alone or in combination. In one embodiment, the cylinder comprises another output port (503) leading to an analyzer for measuring the amount of anesthetic in the vented stream. In one embodiment, a feedback control loop is established for maintaining a constant anesthetic concentration in the gas, or diverting the flow thereby bypassing the anesthetic gas scavenging means.

In one embodiment, the methods of providing the anesthetic gas described herein further comprising a step of determining an amount of anesthetic to be introduced based on a measurement taken using a monitor or gauge of a current amount of volatile gas being circulated to the subject. In another embodiment, the the monitor gauge is disposed between the output port (503) of the volatile gas scavenging means (500) and the output port (22) of the rebreathing device (10). In another embodiment, the amount of anesthetic gas in the inspirated air is controlled by the flowrate of the anesthetic gas through the volatile gas scavenging means. (500). In one embodiment, the methods of providing an anesthetic to a subject in need thereof as described herein, further comprises the step of recovering the volatile gas anesthetic after the step of absorbing the volatile gas anesthetic.

In one embodiment, reclaiming the anesthetic gas comprises a cryogenic method may be used following thermal desorption of the volatile gas from the scavenging means described herein. In another embodiment, thermal desorption is followed by preparatory affinity chromatography to separate the anesthetic gas.

In one embodiment, excess gas is released from the system with a relief/diverter valve (50). In another embodiment, relief of excess gas in the methods described herein is carried out automatically when a monitor or gauge measuring a circulating gas pressure exceeds a predetermined threshold level.

In one embodiment, oxygen uptake, or consumption is independent of the choice of anesthetic agent used and is based on the subject's body weight being about 10×weight^{0.75} ml/min; for example, in one embodiment 270 ml/min for an 81 kg patient. Accordingly, in one embodiment, the methods described herein further comprise the step of introducing oxygen into the anesthetic gas supply. In another embodiment, introducing oxygen to the carrier gas in the methods described herein permits a small quantity of the anesthetic gas, such as sevoflurane in one embodiment, or desflurane in another embodiment to reside in the subject, rather than being carried out as waste anesthetic gas. In one embodiment, adding oxygen as described herein permits use with oxygen sources of limited capacity, such as compact oxygen cylinders and oxygen concentrators in certain embodiments. This makes use of the device and methods described herein in ad hoc anesthetizing locations more practical. In one embodiment, the amount of oxygen introduced into the apparatus approximates the metabolic oxygen consumption of the subject.

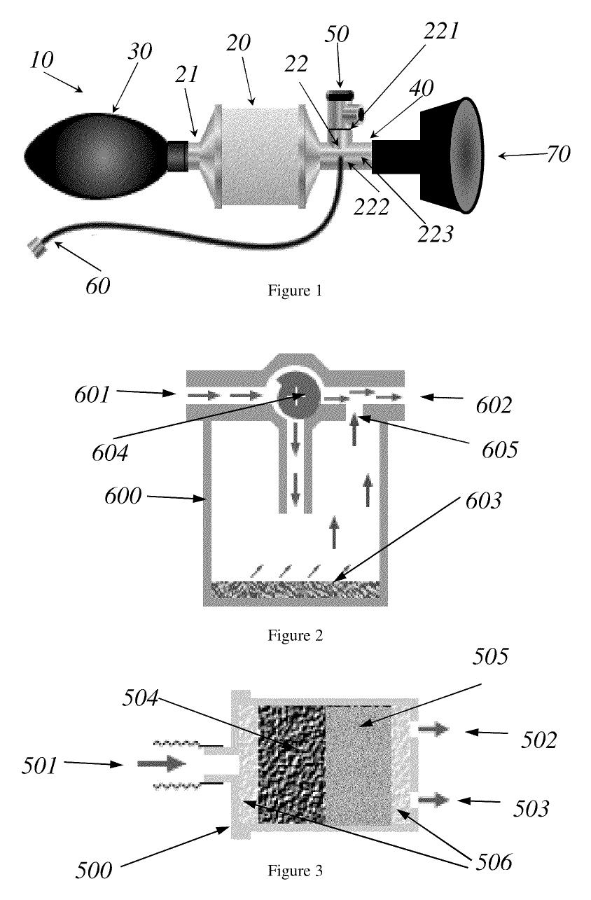

Turning to FIG. 1, showing an apparatus (10) comprising: a canister (20) having an input lumen port (21) and an output lumen port (22), wherein the canister comprises a CO₂ scavenging means, the input lumen port (21) operably linked to a breathing bag (30) and the output lumen port (22) having three openings therein; an axial opening (223), a first radially disposed opening (21), and a second radially disposed opening (222), wherein the first and second radially disposed openings are diametrically opposed; a flow diverting/relief valve (50) operably linked to the first radial opening (221) in the output lumen port (22); an anesthetic gas supply (60), operably linked to the second radially disposed opening (222) in the output lumen port (22); a subject interface means (70), operably linked to the axial opening (223); and a volatile gas scavenging means (500) having a proximal end (501) and a distal end (502), the proximal end being operably coupled to the diverter valve (50); and the distal end operably linked to the output lumen port (22) of the apparatus (10).

In one embodiment, the subject interface means (70), operably linked to the axial opening (223), is capable of affecting a mandibular advancement on the jaw of the subject while supporting continuous positive pressure. In another embodiment, the subject interface means capable of affecting a mandibular advancement while supporting continuous positive pressure comprises: a mandibular interface; a maxilar interface: and a midsection disposed therebetween, the midsection comprising an intubation port and a connection port capable of being coupled to a device for delivering positive airway pressure and wherein the vertical alignment of the mandibular interface relative to the maxilar interface effects a mandibular advancement. An embodiment of the subject interface means capable of affecting a mandibular advancement is described in U.S. patent application Ser. No. 12/247,938 incoporated herein by reference in its entirety.

In another embodiment, the subject interface means (70) is a facemask, an endotracheal tube or a laryngeal mask.

In one embodiment, the anesthetic gas source operably linked to the anesthetic gas supply is an anesthetic vaporizer (600). Turning now to FIG. 2, showing an embodiment of a vaporizer capable of being used with the apparatus described herein, affecting the methods provided herein. FIG. 2 shows the vaporizer (600), having an input port (601) wherein a carrier gas, such as air in one embodiment, or air enriched with Oxygen or other gasses in other embodiments is vented at a predetermined flowrate, temperature and relative humidity into a direction valve (605) which diverts the carrier above a reservoir of the anesthetic gas in a liquid state, allowing equilibration of the liquid anesthetic gas vapors into the carrier gas and into an exhaust vent (606) and through the output port (602) and into the fresh anesthetic supply line (60). In certain embodiment, the vaporizer is temperature controlled. In another embodiment, the diverter valve is self actuated, operably linked to a measurement gauge and monitors controlling the amount of anesthetic gas being incoporated into the carrier gas.

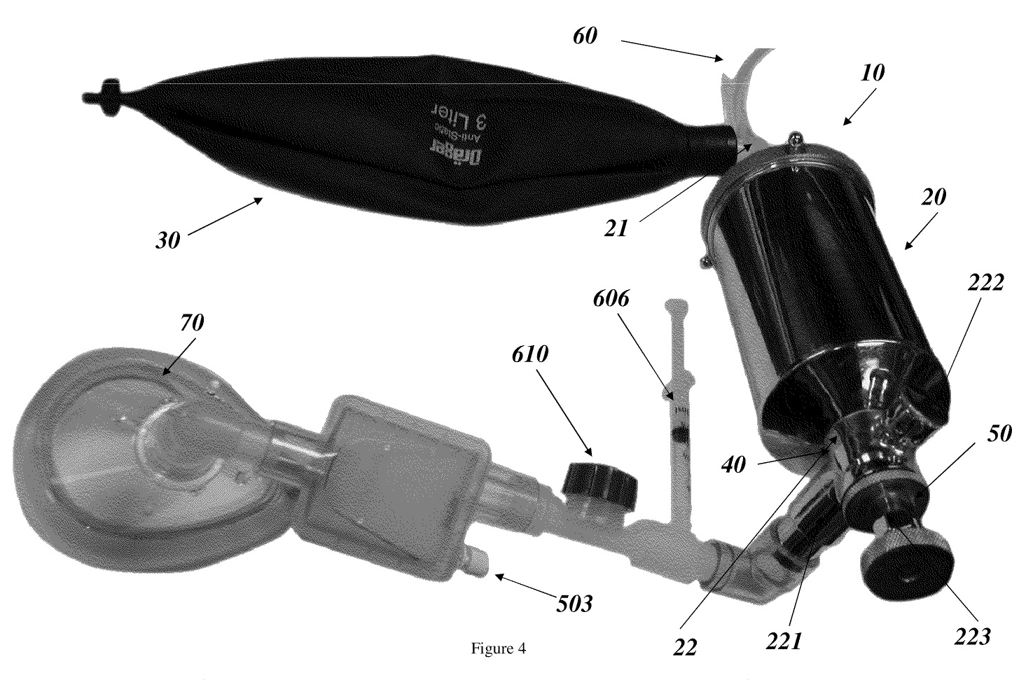

Turning now to FIG. 4, showing another embodiment of the apparatus used in the methods described herein and wherein the apparatus comprises: a canister (20) having an input lumen port (21) and an output lumen port (22), wherein the canister (20) comprises a CO₂ scavenging means, the input lumen port (21) operably linked to a breathing bag (30) and the output lumen port (22) having three openings therein; an axial opening (223), a first radially disposed opening (221), and a second radially disposed opening (222), wherein the first and second radially disposed openings are diametrically opposed; a flow diverting/relief valve (50) operably linked to the axial opening (223) in the output lumen port (22); an anesthetic gas supply (60), operably linked to the input lumen port (21); a subject interface means (70), operably linked to the first radial opening (221), with a volatile gas scavenging means (500) disposed therebetween. In one embodiment, the anesthetic is administered as a liquid, using injection device (60) into the apparatus circuit. In one embodiment, a sampling port (503) is disposed between the subject interface means (70) and the anesthetic supply means (60) for in-line analysis of the anesthetic content. Also shown in FIG. 4, is another gas entry port (610), such as Oxygen as described hereinabove.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method for administering an anesthetic to a patient, the method comprising:
a. streaming the anesthetic into an apparatus comprising.
i. a canister having an input lumen port and an output lumen port, wherein the canister comprises a CO₂ scavenging means, the input lumen port operably linked to a breathing bag and the output lumen port having three openings therein; an axial opening, a first radially disposed opening, and a second radially disposed opening, wherein the first and second radially disposed openings are diametrically opposed;
ii. a flow diverting/relief valve operably linked to the first radial opening in the output lumen port;
iii. an anesthetic gas supply, operably linked to the second radially disposed opening in the output lumen port;
iv. a subject interface means, operably linked to the axial opening;
v. and a volatile gas scavenging means having a proximal end and a distal end, the proximal end being operably coupled to the diverter valve; and the distal end operably linked to the output port of the apparatus;
b. venting the anesthetic gas to the output port of the apparatus for inspiration by the subject;
c. venting expirated air to the CO₂ scavenging means of the apparatus, thereby cleaning the expirated air of CO₂ to be re-circulated to the subject; and
d. diverting expirated air to a volatile gas scavenging means, thereby absorbing at least some of the volatile anesthetic gas in the exhaled air.

2. The method of claim 1, wherein the anesthetic gas comprises Sevofluorane.

3. The method of claim 2, wherein the Sevofluorane is administered to anesthetize a subject for a cardioversion, or other brief painful procedure.

4. The method of claim 1, wherein the Carbon Dioxide (CO₂) scavenging means comprises soda lime.

5. The method of claim 1, wherein the volatile gas scavenging means comprises activated charcoal, Tenax or a combination thereof.

6. The method of claim 1, whereby the step of diverting is bypassed.

7. The method of claim 1, further comprising a step of determining an amount of anesthetic to be introduced based on a measurement taken using a monitor or gauge of a current amount of volatile gas being circulated to the subject.

8. The method of claim 1, comprising venting excess gas via a pressure-relief valve.

9. The method of claim 1, comprising automatically venting gas when a monitor or gauge measures a circulating gas pressure exceeding a predetermined threshold level.

10. The method of claim 1, wherein the anesthetic gas source is an anesthetic vaporizer

11. The method of claim 7, whereby the amount of anesthetic gas in the inspirated air is controlled by the flowrate of the anesthetic gas through the volatile gas scavenging means.

12. The method of claim 1, further comprising the step of recovering the volatile gas anesthetic after the step of absorbing the volatile gas anesthetic.

13. The method of claim 1, further comprising the step of introducing oxygen into the anesthetic gas supply.

14. The method of claim 13, whereby the amount of oxygen introduced into the apparatus approximates the metabolic oxygen consumption of the subject.

15. The method of claim 1, whereby the subject interface means is capable of affecting a mandibular advancement on the jaw of the subject while supporting continuous positive pressure.

16. The method of claim 1, whereby the subject interface means is a facemask, an endotracheal tube or a laryngeal mask

17. The method of claim 15, whereby the subject interface means capable of affecting a mandibular advancement while supporting continuous positive pressure comprises: a mandibular interface; a maxilar interface; and a midsection disposed therebetween, the midsection comprising an intubation port and a connection port capable of being coupled to a device for delivering positive airway pressure and wherein the vertical alignment of the mandibular interface relative to the maxilar interface effects a mandibular advancement.

18. An apparatus comprising:
a. a canister having an input lumen port and an output lumen port, wherein the canister comprises a CO₂ scavenging means, the input lumen port operably linked to a breathing bag and the output lumen port having three openings therein; an axial opening, a first radially disposed opening, and a second radially disposed opening, wherein the first and second radially disposed openings are diametrically opposed;
b. a flow diverting/relief valve operably linked to the first radial opening in the output lumen port;
c. an anesthetic gas supply, operably linked to the second radially disposed opening in the output lumen port;
d. a subject interface means, operably linked to the axial opening;
e. and a volatile gas scavenging means having a proximal end and a distal end, the proximal end being operably coupled to the diverter valve; and the distal end operably linked to the output port of the apparatus.

19. The apparatus of claim 18, wherein the subject interface means is capable of affecting a mandibular advancement on the jaw of the subject while supporting continuous positive pressure.

20. The apparatus of claim 19, wherein the subject interface means capable of affecting a mandibular advancement while supporting continuous positive pressure comprises: a mandibular interface; a maxilar interface; and a midsection disposed therebetween, the midsection comprising an intubation port and a connection port capable of being coupled to a device for delivering positive airway pressure and wherein the vertical alignment of the mandibular interface relative to the maxilar interface effects a mandibular advancement.
